(19) United States
(12) Patent Application Publication
(10) Pub. No.: US 2021/0012869 A1
(43) Pub. Date: Jan. 14, 2021

(54) Systems and Methods for Real-Time Health Certification Using Point-of-Care Data

(71) Applicant: Biosafety Diagnostics, Inc., Hoboken, NJ (US)

(72) Inventors: Michael Kotlarz, Short Hills, NJ (US); Keith Barksdale, Hoboken, NJ (US)

(73) Assignee: Biosafety Diagnostics, Inc., Hoboken, NJ (US)

(21) Appl. No.: 17/037,563

(22) Filed: Sep. 29, 2020

Related U.S. Application Data
(60) Provisional application No. 63/055,201, filed on Jul. 22, 2020.

(51) Int. Cl. G16H 15/00; G16H 10/60; G16H 10/40; G06Q 30/00; G16H 50/70; G16H 50/30; G16H 70/20; G06F 16/25; G16B 45/00; G16B 40/00

(52) U.S. Cl. CPC G16H 15/00; G16H 10/60; G16H 10/40; G06Q 30/0185; G16H 50/70; G06K 19/06037; G16H 70/20; G06F 16/258; G16B 45/00; G16B 40/00; G16H 50/30

(57) ABSTRACT

A health pass system for COVID-19 is provided. The system includes a database including information on a plurality of individuals. The database is configured to receive point-of-care data relating to the plurality of individuals, the point-of-care data including at least one medical test result for COVID-19 antibodies for at least one of the plurality of individuals. The system includes a processing device configured to analyze the point-of-care data and generate a report. The system includes means for transmitting the report to a mobile electronic device of at least one of the plurality of individuals. The system includes means for displaying the report with a user interface of the mobile electronic device in the form of a real-time health certification.

This application is a United States Non-Provisional Patent Application which claims priority to U.S. Provisional Patent Application No. 63/055,201, filed on Jul. 22, 2020, the entire contents of the foregoing patent application are hereby expressly incorporated by reference.

### BACKGROUND

### Technical Field

The present disclosure relates generally to the field of computer-based health tracking and data systems. More specifically, the present disclosure relates to systems and methods for real-time health certification using point-of-care data.

### Related Art

In today's world, real-time tracking of the health of individuals is gaining increased importance. Recently, global health pandemics (such as the current coronavirus pandemic) have made the detection and tracking of individuals who have tested positive for illnesses particularly important in assisting with curtailing the spread of such illnesses. Moreover, the ability to assess, in real time, the health risk posed by individuals is likely to be highly useful in connection with activities, events, transactions, and locations (both physical and virtual) where biological risk may be a concern.

Point-of-care health data represents an important source of information that can be used to ascertain the health risks of individuals. Examples of such data include, but are not limited to, manual and automated laboratory test results, medical treatment and/or diagnosis results, private testing results, and other sources of point-of-care health data. A particular technological problem with such point-of-care health data is that the information is often stored in proprietary, incompatible data formats. Moreover, currently there is not an acceptable real-time system that can communicate with disparate point-of-care health data systems, consolidate the data into a consolidated, centralized, real-time database, and processes the consolidated data rapidly in order to provide a real-time risk assessment of whether a particular individual suffers from a biological risk that may make participation in an activity, event, or transaction, or attendance at a particular location, inadvisable from a public health and safety perspective. For example, in the current coronavirus pandemic, what is lacking is a real-time system that can rapidly ascertain the risk level of a person who may be suffering from coronavirus using point-of-care data stored in various, disparate, and often incompatible data platforms formats, and which can be used to monitor and/or regulate such person's future activities. Still further, there is currently a need for a real-time system that can be utilized to electronically certify that a person is approved to engage in an activity and/or to be present at a location, using point-of-care data.

Accordingly, what would be desirable, but has not yet been provided, are systems and methods for real-time health certification using point-of-care data which solve the foregoing and other needs.

### SUMMARY

This present disclosure relates to systems and methods for real-time health certification using point-of-care data. The system includes a consolidated, centralized, real-time database that stores point-of-care data in a common storage format, and a certification server in communication with the database. The point-of-care data could include, but is not limited to, manual and automatic laboratory test results, medical (e.g., hospital) patient information, private test results, and mandated test results. The certification server processes the real-time database and calculates a risk score for an individual, and can generate an electronic report for the patient in response to an electronic query, which includes the risk score for the patient, an indication of whether the individual is currently suffering from an illness, and optionally, a data symbol (e.g., a QR code) which electronically encodes the report and can be used to electronically certify that the individual is authorized to engage in one or more events or activities, access one or more services or devices, and/or attend one or more locations. Optionally, the report can be accessed by computer systems maintained by one or more third-parties such as corporate entities, travel entities, electronic medical records custodians, educational entities, healthcare entities, medical research entities, governmental entities, and/or other third-party entities, using one or more customized application programming interfaces (APIs).

The present disclosure relates to a health pass system for COVID-19. The system includes a database including information on a plurality of individuals. The database is configured to receive point-of-care data relating to the plurality of individuals. The point-of-care data includes at least one medical test result for COVID-19 antibodies for at least one of the plurality of individuals. The system includes a processing device configured to analyze the point-of-care data and generate a report. The system includes means for transmitting the report to a mobile electronic device of at least one of the plurality of individuals. The system includes means for displaying the report with a user interface of the mobile electronic device in the form of a real-time health certification. The report includes the at least one medical test result for COVID-19 antibodies for the at least one individual, and an indication of a positive or negative result of the at least one medical test result for COVID-19 antibodies. The report can include a code (e.g., a QR code, a bar code, or the like) capable of being scanned to display information associated with the report on another user interface or an electronic device.

In some embodiments, the report can include a photograph of the individual. In some embodiments, the report can include a unique alphanumerical identification associated with the individual. In some embodiments, the unique alphanumerical identification can be used to access the report. In some embodiments, the report can include, e.g., a name of the individual, a type of test performed for the at least one medical test result, a date of the test performed for the at least one medical test result, combinations thereof, or the like. The point-of-care data can include at least one of manual laboratory test results, automatic laboratory test results, medical information, private test results, or mandated test results. The point-of-care data can include information relating to or derived from at least one of DNA, antibodies, psychological evaluation, existing conditions, medical history, plasma, blood composition, saliva, bodily fluid, stool, bone marrow, enzymes, glucose, cholesterol, immunology, gastric fluid, kidney function, inulin, pregnancy, toxicology, urinalysis, thyroid function, genetic testing, or skin testing.

The at least one medical test result for COVID-19 antibodies can include results for IGG+ and IGM− antibodies. In some embodiments, the database can be configured to receive the point-of-care data from medical tests in real-time. In some embodiments, the database can be configured to receive the point-of-care data from already administered medical tests. In some embodiments, the database can be configured to receive the point-of-care data from an automated testing system or a photograph of a medical test result received as input.

The processing device can be configured to consolidate the point-of-care data and convert the point-of-care data into a common storage format having a unified data structure. The processing device can be configured to generate a risk score based on analysis of the point-of-care data. The risk score can indicate one or more biological risks associated with the plurality of individuals. In some embodiments, the processing device can be configured to generate the risk score based on the point-of-care data and medical guidelines including thresholds for the point-of-care data regarding biological illness levels or risks. In such embodiments, the processing device can be configured to automatically update the risk score based on real-time updated medical guidelines.

In some embodiments, the processing device can be configured to generate the risk score based on the point-of-care data, prior conditions, demographics, and data collected at a point of presence. The report can include the risk score. In some embodiments, based on the generated risk score, the report can include a designation of safe or note safe for the at least one of the plurality of individuals (as an alternative to or in combination with the risk score).

The present disclosure relates to a method of generating a health pass for COVID-19. The method includes receiving at a database information a plurality of individuals. The method includes receiving at the database point-of-care data relating to the plurality of individuals. The point-of-care data includes at least one medical test result for COVID-19 antibodies for at least one of the plurality of individuals. The method includes analyzing the point-of-care data and generating a report with a processing device. The method includes transmitting the report to a mobile electronic device of at least one of the plurality of individuals. The method includes displaying the report with a user interface of the mobile electronic device in the form of a real-time health certification. The report includes the at least one medical test result for COVID-19 antibodies for the at least one individual, an indication of a positive or negative result of the at least one medical test result for COVID-19 antibodies, and a code (e.g., a QR code, a bar code, or the like) capable of being scanned to display information associated with the report on another user interface or an electronic device.

In some embodiments, the report can include, e.g., a photograph of the individual, a name of the individual, a type of test performed for the at least one medical test result, a date of the test performed for the at least one medical test result, combinations thereof, or the like. In some embodiments, the at least one medical test result for COVID-19 antibodies includes results for IGG+ and IGM− antibodies.

The present disclosure relates to non-transitory computer-readable medium storing instructions at least for generating a health pass for COVID-19 that are executable by a processing device. Execution of the instructions by the processing device causes the processing device to receive at a database information a plurality of individuals. Execution of the instructions by the processing device causes the processing device to receive at the database point-of-care data relating to the plurality of individuals. The point-of-care data includes at least one medical test result for COVID-19 antibodies for at least one of the plurality of individuals. Execution of the instructions by the processing device causes the processing device to analyze the point-of-care data and generate a report. Execution of the instructions by the processing device causes the processing device to transmit the report to a mobile electronic device of at least one of the plurality of individuals. Execution of the instructions by the processing device causes the processing device to display the report with a user interface of the mobile electronic device in the form of a real-time health certification. The report includes the at least one medical test result for COVID-19 antibodies for the at least one individual, an indication of a positive or negative result of the at least one medical test result for COVID-19 antibodies, and a code (e.g., a QR code, a bar code, or the like) capable of being scanned to display information associated with the report on another user interface or an electronic device. In some embodiments, the report can include, e.g., a photograph of the individual, a name of the individual, a type of test performed for the at least one medical test result, a date of the test performed for the at least one medical test result, combinations thereof, or the like.

### BRIEF DESCRIPTION OF THE DRAWINGS

The foregoing features of the invention will be apparent from the following Detailed Description of the Invention, taken in connection with the accompanying drawings, in which:

FIG. 1 is a diagram illustrating hardware and software components of the system of the present disclosure;

FIG. 2 is a diagram illustrating the system of FIG. 1 in greater detail;

FIG. 3 is a diagram illustrating an example of an electronic health certification report capable of being generated by the system of the present disclosure;

FIG. 4 is a diagram illustrating another example of an electronic health certification report capable of being generated by the system of the present disclosure;

FIG. 5 is a diagram illustrating a visualization tool capable of being generated by the system of the present disclosure, for graphically depicting the locations of individuals at a venue; and

FIG. 6 is a flowchart illustrating processing steps carried out by the system for calculating a health risk score for an individual.

### DETAILED DESCRIPTION

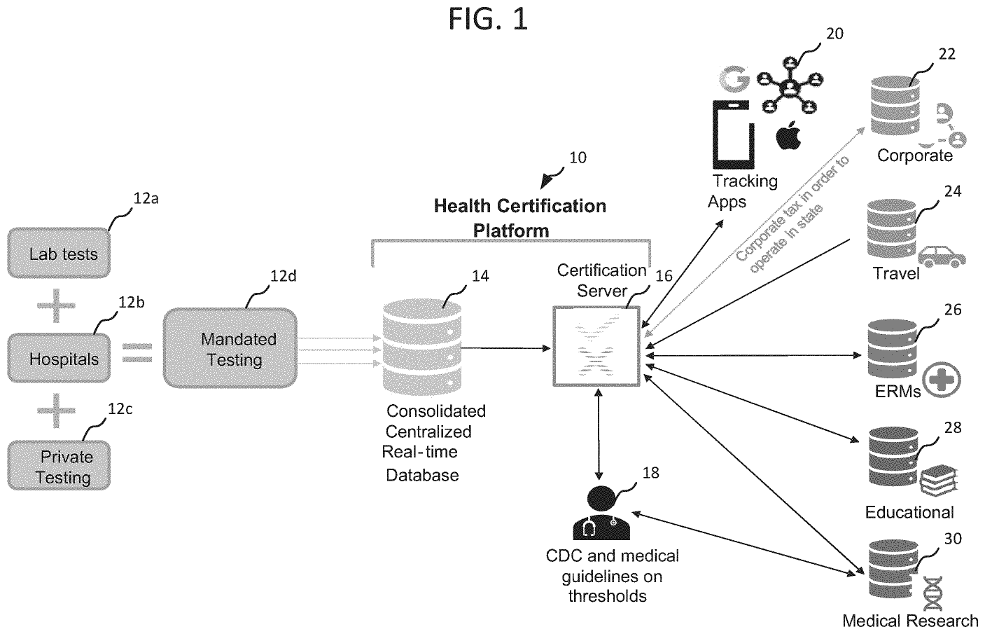

The present disclosure relates to systems and methods for real-time health certification using point-of-care data, as described in detail below in connection with FIGS. 1-6.

FIG. 1 is a diagram illustrating hardware and software components of the health certification system 10 (hereinafter “system 10”) of the present disclosure. Components 12a-12d of the system 10 represent a “testing” phase, components 14-18 of the system 10 represent a “tracking” phase, and components 20-30 represent a “tracing” phase, as will be described in greater detail below. In the “testing” phase (e.g., a collection phase), point-of-care data from a variety of sources can be collected in real-time. The point-of-care data could include, but is not limited to, manual and automatic laboratory test results (e.g., lab tests 12a), medical (e.g., hospital) patient information (e.g., hospital testing 12b), private test results (e.g., private testing 12c), and mandated test results (e.g., mandated testing 12d).

The point-of-care data can be collected from a test in real-time, and/or from an already administered test. As an example the lab tests 12a, hospital testing 12b and private testing 12c can be used to collect data on a variety of individuals, e.g., first responders, teachers, students, employees, citizens, combinations thereof, or the like. As a further example, the mandated testing 12d can be used to collect data focused on specific biological illnesses or infectious diseases. Such mandated testing 12d can include, but is not limited to, COVID-19, SARS, MERS, measles, or the like. The types of biological information that can be used for collection of the point-of-care data can include, but is not limited to, DNA, antibodies, psychological evaluation data, existing conditions, medical history, plasma, blood composition, plasma, saliva, any bodily fluid, urine, stool, bone marrow, enzymes, glucose, cholesterol, immunology, gastric fluid, kidney function, inulin, pregnancy, toxicology, urinalysis, thyroid function, genetic testing, skin testing, or the like.

The point-of-care data can be collected through, e.g., manual input into the system 10, wireless computer protocols, LIS servers, HL7 diagnostic protocols, HIPAA compliant database queries, batch processing from employment and medical records, insurer records, any other means of digital entry, or the like. In some embodiments, the system 10 can use machine vision to read and analyze the results of manual tests given the make and model of the test, and populates the data into the system 10. For example, if a cell phone photograph is received as input by the system 10 for a manual test, the system 10 can use machine vision to retrieve details of the point-of-care data from the test for further analysis. The system 10 can also read or retrieve the point-of-care information directly from an automated testing system.

In some embodiments, each individual for whom data is collected can complete a registration form for consent of participation in the system 10. Such registration can result in a unique alphanumerical identification (e.g., a personal or person ID) associated with the specific individual for collecting and maintaining point-of-care data for the individual over time. The system 10 can aggregate the data from the “testing” phase into a consolidated centralized real-time database 14 from all testing locations. In aggregating the data, the system 10 can convert the point-of-care data into a common storage format to ensure all data can be used effectively to evaluate the biological risk of the individual associated with each person ID. In particular, the system 10 can aggregate various machine or lab results to centralize all test results for the individual, and consolidates and translates various datatypes and formats into unified data structure. The system 10 can thereby operate and integrate with any testing system. In some embodiments, the system 10 can process 1,000,000 or more data points/second.

A certification server 16 can receive as input the consolidated point-of-care data from the database 14, and generates a risk score for each individual based on the point-of-care data. The generated risk score can also be tied to the person ID based on the person ID used for the consolidated point-of-care data. The system 10 can receive as input at the certification server 16 up-to-date guidelines 18 in real-time to assist in generation of the risk score. Such guidelines 18 can include, e.g., CDC and/or medical guidelines on thresholds for point-of-care data to evaluate the biological illness level or risk of an individual. The guidelines 18 can be updated in real-time as a set of rules for the risk score generation based on the ongoing and changing scientific understanding of the biological risk involved. Operation of the certification server 16 is thereby dynamic based on ongoing changed in the guidelines 18, as well as updated testing for the individual received at the database 14 in real-time.

In addition to using the testing data to evaluate and determine the risk score for the individual, the system 10 can receive as input an aggregate from other systems to assist in the analysis of the biological risk. The additional data can include, e.g., prior conditions, demographics, data collected at the point of presence, combinations thereof, or the like, and can be used to assist in rendering a risk score or rating. The risk scores can be output by the system 10 as safety levels, e.g., with level 1 being the safest, and each increasing level representing an additional level of risk associated with the individual.

For example, with respect to COVID-19, the certification server 16 can used antibody testing data for the analysis, as well as supplemental factors. In one instance, the certification server 16 analysis can be as follows: COVID-19 IGG+ with IGG index >20° and no prior conditions, IGM negative with index <0.5, body temperature <100° F., to output a safety level 1 or safest level for the individual. In another instance, the certification server 16 analysis can be as follows: COVID-19 IGG+ with IGG index >20 and IGG <0.80 and no prior conditions, IGM negative with index <1, body temperature <99° F., to output a safety level of 2 or the next safest level for the individual. The safety level association with the person ID can be automatically updated in real-time based on additional testing data received by the system 10 for the individual and/or based on updated guidelines 18 regarding biological risks associated with the individual. It should be understood that the certification server 16 analysis can include several other levels of supplemental data based on different inputs for determination of the risk score.

As a further example, with COVID-19, the system 10 can be initially programmed to a very high level of IGG antibodies (IGG+) and a very low level of IGM Antibodies (IGM−) to denote a low COVID-19 risk level, and borderline immunity. Such antibody levels can be used as thresholds for analysis by the certification server 16. As medical or governmental regulatory standards change (i.e., guidelines 18) and as the system 10 learns more about the disease through the updated guidelines 18, the thresholds of risk levels can be adjusted and applied to the existing dataset. The system 10 can thereby continuously or substantially continuously update the risk score for individuals based on the updated guidelines 18 based on testing data previously received by the system 10, reducing the need to re-test individuals if the data is available. In some embodiments, such updating by the system 10 can be based on the type of test taken previously by the individual. For example, in some instances, a re-test may be necessary to determine the current level of antibodies of the individual. The continuously or substantially continuously updating of the testing data can be similarly used for any biological data being recorded and aggregated, including other non-biological inputs that may inform the risk score determination. For example, non-biologic inputs that may affect the risk score determination can include place of origin and/or demographics where a known biologic risk is higher.

The risk score generated for each individual can be electronically stored in the certification server 16. In some embodiments, a separate biological risk level database can receive as input the risk score generated by the certification server 16, and the risk levels or risk scores electronically stored in the risk level database can be automatically updated in real-time based on continued analysis of the certification server 16. The risk level database can thereby be created using the biological factor raw data inputs and the logic used by the certification server 16 to create and calculate the risk level. The person ID number for each respective individual can be electronically tied to their corresponding calculated risk score. The person ID can be used as a unique electronic element or identifier to access or transact physically or electronically with subsequent queries for the risk score of the individuals, such as queries by physical/virtual venues, physical/virtual services, places of business, transit, education, commerce of any type, combinations thereof, or the like. The risk score and any data associated with the individual can be securely encrypted and authenticated in the respective database(s).

In some embodiments, the individual associated with the encrypted unique person ID number can be provided with a selection to allow the entity/user to electronically decide who has access to the risk score information and for what purpose. For example, the individual can selectively choose who has access to the risk score and associated information on a case-by-case basis. In some embodiments, the individual can provide a one-time confirmation to allow all future access to the risk score data until the individual decides to manually opt out of such access.

The score level and, optionally, additional data associated with the risk score determination, can be provided to a variety of requestors to gauge the risk level of the individual. As non-limiting examples, FIG. 1 depicts such requestors as the following industries or entities: tracking applications 20, corporate 22, travel 24, enterprise risk management (ERMs) 26, educational 28, medical research 30, combinations thereof, or the like. One or more customized application programming interfaces (APIs) can be used for retrieval and/or transmission of the data associated with the risk score based on queries from one or more individuals and/or entities. In some embodiments, each risk score can be retrieved in real-time, encrypted, with time-stamped verification, through a series of levels of detail appropriate to the authenticated requested party. An API with any customizable set of information interested to the authenticated requestor can be provided, depending on the requestor. For example, a request by an employer or a university may have more detail, such as the risk score and additional data that indicates the reasons for the particular risk score. As a further example, a request from an e-commerce entity can be merely “safe” or “not safe” without providing a specific risk score or detail about the risk score determination.

Access and transmission of the risk score to the endpoints requesting the information can be performed in a highly secure and dynamic manner. Each endpoint can be authorized by the individual to gain access to the appropriate API level of information according to the need of the requestor. Such authorization can be provided on a case-by-case basis (e.g., with the individual receiving a notification upon each request with an option to permit or deny access), or can be provided by the individual during registration for the system 10. Examples of automated decisions where requests for the risk score are initiated can include, e.g., allowing a transaction to occur, opening a door, or accessing a space. In such instances, prior to allowing the transaction, opening the door, or permitting access to a space, the endpoint can automatically request the risk score status of the individual and, if the risk score is below a predetermined threshold (indicating that the individual is safe), the endpoint can permit the desired action to occur. If the risk score is above a predetermined threshold (indicating that the individual is not safe), the desired action can be denied and a message can be displayed to the individual indicating the reason for such denial.

For example, an individual can order an UBER® vehicle and, before UBER® provisions a vehicle, UBER® can query the system 10 to determine the biological risk score of the potential passenger. If the risk score is above a predetermined threshold, UBER® can alert the individual that a vehicle cannot be provided. If the risk score is below a predetermined threshold, UBER® can proceed with providing a vehicle to the individual and, optionally, can indicate to the individual that the risk score is acceptable.

A similar process can be performed for airline tickets, sports tickets, restaurant reservations, or any number of situations where contact and crowds could pose a risk. In the travel industry, areas involving airline ticketing, boarding pass registration, passport/visa control, train ticketing, ticket scanning, or the like, can incorporate the system 10 to obtain the risk score of individuals and, based on the risk scores, determine whether the individual should be permitted in the respective areas or on the vehicle for transport. Similarly, transit areas (such as bus ticketing, bus access, subway ticketing, and subway access) can determine the score level of individuals prior to permitting admission.

Sports venues can query for risk scores for individuals prior to permitting ticket purchase and again prior to stadium/venue entry based on depiction or association of a person ID with the ticket. The employment industry can integrate the risk score determination by the system 10 with employee timeclock systems to check the employee's risk score each time a clock-in procedure or entry into an establishment occurs. The education industry can integrate the risk score determination by the system 10 with students and faculty management systems such that schools, colleges and universities can better meet the specific treatment and educational needs of their students. For example, a student who is considered not yet safe based on the associated risk score may be offered virtual education in place of in person education until the student is certified as safe by the system 10. As a further example, a faculty member who is considered not safe based on the associated risk score may be asked to remain off the educational facility premises until the faculty member is certified as safe by the system 10. Physical entry of individuals and/or vehicles into a location having any existing gate and/or security infrastructure can also incorporate the system 10 to determine the biological safety level of the individual prior to permitting entry. The system 10 can be integrated into Enterprise Resource Planning (ERP) and/or point-of-sale (POS) systems. With such integration, companies such as SQUARE®, who offer fully integrated connected POS systems, can offer the ability to make their small business customers and their clients safe.

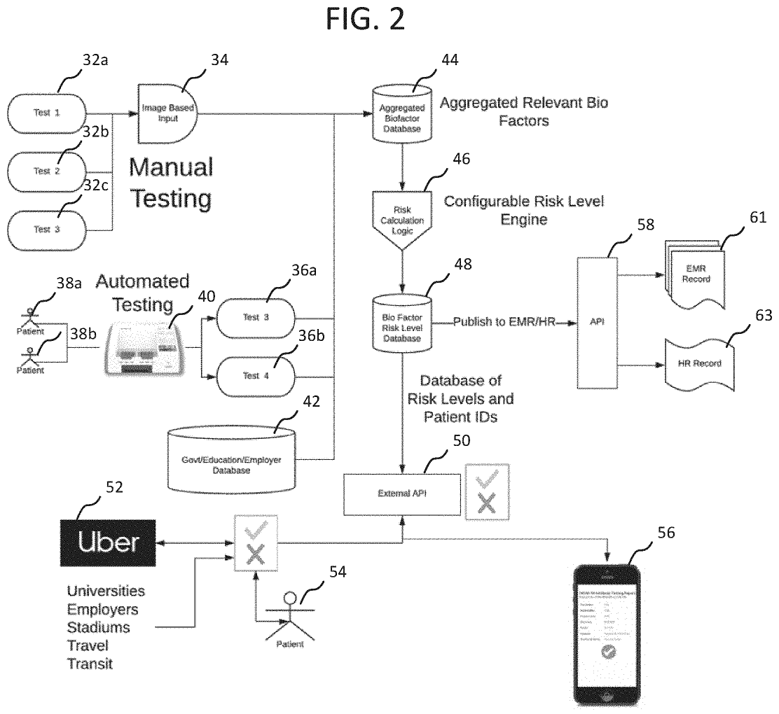

FIG. 2 is a diagram illustrating the system 10 of FIG. 1 in greater detail. As noted above, a variety of manual and/or automated testing can transmit point-of-care data to the system 10. Such data can be transmitted to the system 10 in real-time to ensure the most up-to-date information is being analyzed by the system 10. However, the system 10 can also receive as input point-of-care data for testing performed in the past, and can take into account the older point-of-care data in the risk score analysis. For manual testing, any number of tests 32a, 32b, 32c can be performed, and images 34 of the test results can be used as input into the system 10. The system 10 can in turn analyze the images 34 to determine the relevant data for extraction and further analysis. Automated testing can be performed for any number of patients 38a, 38b via an automated testing machine 40. For example, the automated testing machine 40 can be a finger prick machine to test for antibodies of the individual. In some embodiments, the finger prick machine can be a small and portable unit that provides results in substantially real-time (e.g., about 10 minutes). Automated results 36a, 36b of the tests can be output by the testing machine 40 and can be received as input by the system 10 for further analysis.

The point-of-care data can be electronically transmitted to an aggregated biofactor database 44 (e.g., database 14) that consolidates the data in a standard or uniform format capable of being used by the system 10. A database 42 maintaining updated medical guidelines for a variety of industries (e.g., government, education, employer, or the like) can be coupled to the system 10 and can be updated in real-time. A configurable risk level engine 46 (partially or fully incorporated in the certification server 16) having risk calculation logic programmed therein can receive as input the point-of-care data and the guidelines from database 42, and determines the risk, and/or risk score associated with each individual. The risk scores can be electronically stored in a biofactor risk level database 48, with each risk score associated with a unique person ID.

In some embodiments, the database 48 can be communicatively coupled to an internal API 58 for transmission of risk scores to EMR records 61 and human resources (HR) records 63. In some embodiments, external APIs 50 can be communicatively coupled to the database 48 to query the database 48 for risk scores associated with individuals. For external APIs 50, the system 10 can output the necessary information based on the type of entity requesting the information. For example, the system 10 can output to a requestor 52 an indication that the individual is safe or not safe. As a further example, the system 10 can output to a requestor via a graphical representation or report on an electronic device 56 a report with the risk score, whether the individual is safe or not safe, and additional data used for determining the risk status of the individual. As a further example, the system 10 can output to the patient 54 their risk status level.

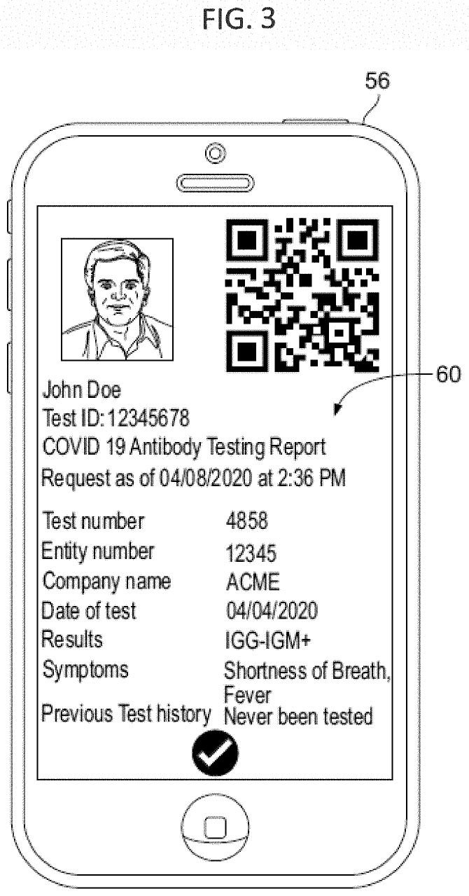

FIG. 3 is a diagram illustrating an electronic health certification report 60 capable of being generated by the system 10 of the present disclosure. The report 60 can be generated and depicted on an electronic device 56 (e.g., a mobile electronic device, a computer, or the like). The report 60 can also be provided in a standard format that can be forwarded or shared with additional parties. The report 60 can include, e.g., a photograph of the individual, the name of the individual, a person or test ID associated with the individual, a description of the type of biologic test performed, a date of request of the report, a test reference number, a requestor entity number, a requestor company name, a date of the biologic test, results of the biologic test, any symptoms felt by the individual, previous biologic test history of the individual, combinations thereof, or the like. In some embodiments, the report 60 can include a quick response (QR) code or barcode that can be scanned to transmit the report 60 to another individual. The report 60 of FIG. 3 indicates that the individual had a COVID-19 antibody test on Apr. 4, 2020, with results for IGG+ and IGM− antibodies, with the individual being asymptomatic at the time of the test, and no previous testing.

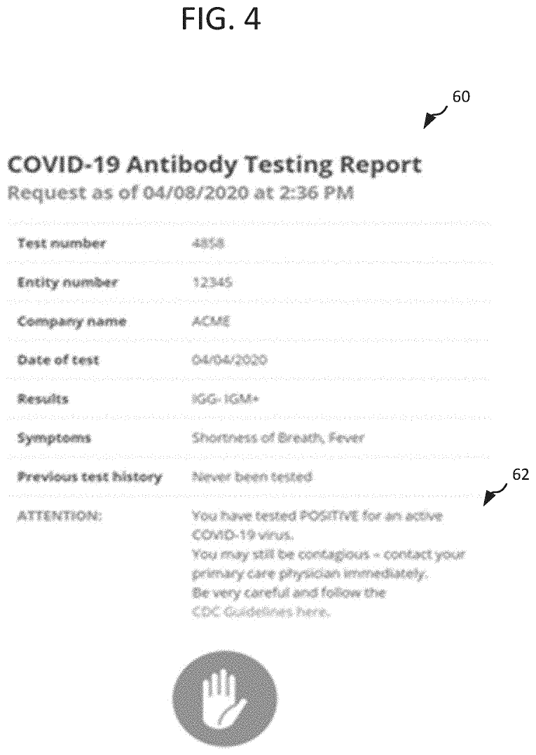

FIG. 4 is a diagram illustrating another electronic health certification report 60 capable of being generated by the system 10 of the present disclosure. The report 60 of FIG. 4 indicates that a COVID-19 antibody test was performed on Apr. 4, 2020, with results for IGG+ and IGM− antibodies. The report 60 further indicates that the patient had shortness of breath and a fever at the time of the test. If the system 10 determines that the individual may be a risk to others based on the tests performed, the report 60 can include an alert or attention section 62 in which important information can be presented. For example, the report 60 of FIG. 4 provides that the patient tested positive for the COVID-19 virus and may still be contagious in the attention section 62. In some instances, the graphic at the bottom of the report 60 can visually indicate whether the individual can proceed with the desired activity (e.g., purchasing a ticket, checking in to a flight, boarding a bus, or the like). In some instances, the attention section 62 can provide information about whether the individual can proceed with the desired activity.

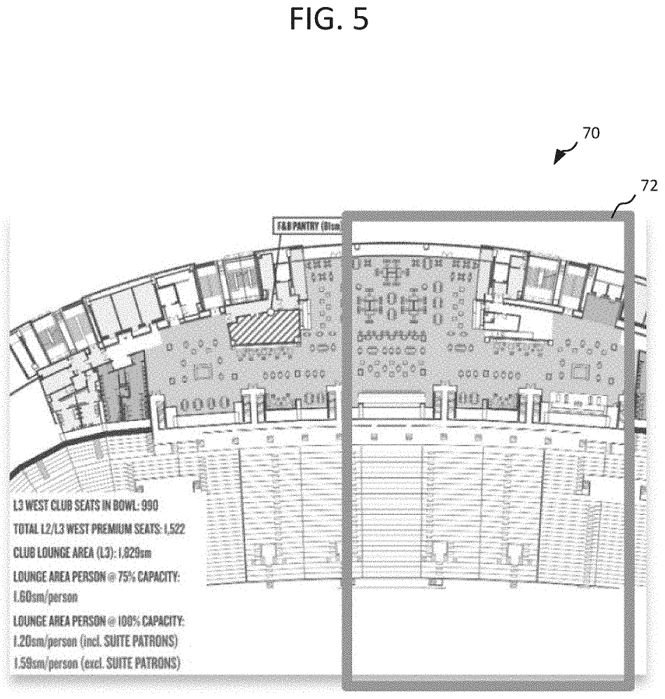

FIG. 5 is a diagram illustrating a visualization tool capable of being generated by the system 10 of the present disclosure, for graphically depicting the locations of individuals at a venue. As an example, FIG. 5 illustrates a partial map 70 of a seating arrangement in a stadium. The system 10 can provide means for selecting a specific section 72 of the interactive map 70 to obtain risk score or level information of individuals associated with the specific section 72. A venue operator can thereby select specific areas of focus on the map to determine the number of individuals in each area and whether one or more of the individuals create a risk to others in the area.

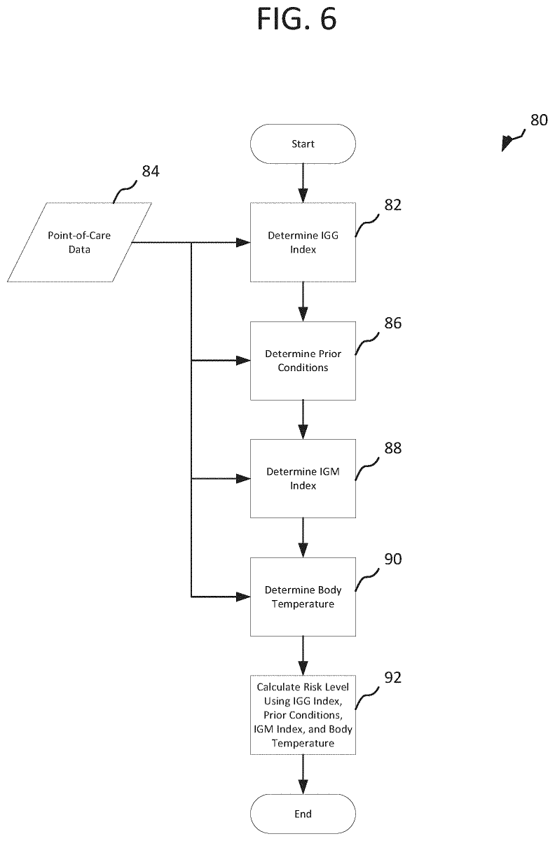

FIG. 6 is a flowchart illustrating processing steps 80 carried out by the system 10 for calculating a health risk of an individual, or a score for an individual. At step 82, the system 10 can determine the IGG antibody index for the individual, at step 86 the system 10 can determine any prior conditions associated with the individual, at step 88 the system 10 can determine an IGM antibody index for the individual, and at step 90 the system 10 determine the body temperature for the individual. Each of steps 82, 86, 88, 90 can receive as input point-of-care data collected at step 84 by the system 10. The point-of-care data can include manual testing and/or automated testing results, both in real-time and previously performed tests. Steps 82, 88 can incorporate medical guidelines associated with biological risks to determine whether the IGG index or the IGM index, respectively, are at levels below or above potential biological risks for the individual. At step 92, the IGG index, prior conditions, IGM index, and body temperature associated with the individual can be used to generate a risk score or level. The risk score or level can be updated at step 92 in a real-time or substantially real-time manner as additional point-of-care data is obtained and/or as medical guidelines are updated.

The system discussed herein therefore provides means for detecting and assigning a risk level for each individual tested based on the point-of-care data, and provides a real-time risk assessment of whether a particular individual suffers from a biological risk that may make participation in an activity, event, or transaction, or attendance at a particular location, inadvisable from a public health and safety perspective. Individuals with low risk levels can be permitted to proceed with participation in the activity, event, or transaction, or attendance at a particular location with minimal risks to the surrounding individuals. Individuals with high risk levels can be prevented from participation in the activity, event, or transaction, or attendance at the particular location to minimize risks to surrounding individuals. Individuals with low risk levels can thereby be permitted to work in safer environments, while ensuring point-of-care data is maintained in an encrypted manner to protect the individual.

The point-of-care data and risk levels can be updated in real-time based on additional testing and updates in medical guidelines. The system can collect data from a variety of sources, allowing for certification of individuals at any global location in real-time. The enrollment process of individuals can also be efficient, providing individuals with an opportunity to decide which entities are permitted to access the risk score associated with the individual and any additional data associated with the risk score.

The testing components of the system can receive point-of-care data from manual and/or automated testing locations, including mandatory testing at approved locations. Such locations can provide testing results indicating the biological levels associated with a variety of biological risks, such as antibodies associated with COVID-19. The tracking components of the system can populate one or more databases with the point-of-care data and medical guidelines, with such data and guidelines used by the system to generate a risk score or level for each individual. Such risk score or level can be used for first responders, students, employees, citizens, combinations thereof, or the like, to determine if such individuals can participate in certain events in environments having other individuals. The risk score or level and associated data can be encrypted to ensure protection to the individual when such data is accessed by healthcare providers, schools, employers, or the like. The tracing components of the system can digitally integrate and correspond with a variety of requestors to provide predetermined amount of information regarding the risk level of the individual. For example, such information can be only safe or not safe, or can provide details of the risk level.

The system discussed herein can be integral to economic re-entry during and after biological crises. For example, federal and state officials can mandate all approved tests to be collected to a secure, centralized, cloud-based database. Federal and state officials can subsequently require all enterprises and employees to be tested. The system can collect and log tests in the centralized database in a uniform format for analysis. The system can digitally certify individuals for re-entry based on CDC and medical guidelines in an updated manner. Results and certifications can be securely submitted electronically to, e.g., student rosters, employee lists, EMRs, Department of Motor Vehicles (DMVs), or the like. Certification results and checkpoints can be integrated throughout the social ecosystems to determine whether individuals can participate in certain events. Economic activity can thereby return to normal with less-restrictive, yet safe guidelines.

The system can be integrated with operations management and public communications systems. For example, the system can include a proprietary set of assets intended to support the development and adoption of the private system/public dashboards. A customized operations management dashboard can be used across the network of professional workforce, distributions, and industries. Public dashboard and communication systems can integrate with private management systems and inspire/power analytics platforms for tracking and tracing of data for individuals. Real-time analytics of tracing for any connections of positive case individuals, transit systems, events, or the like, can be provided in a dashboard user interface, with alert text and updates provided to individuals or entities via the dashboard. The dashboard can provide tools to visualize and narrow positive tests to specific areas (e.g., specific areas of a venue for an event), and based on location, industry and/or demographics. The dashboard can also provide focused notifications and alerts to specific sets of individuals and groups, and provides the ability to narrow the scope of the spread of high-risk individuals.

Having thus described the system and method in detail, it is to be understood that the foregoing description is not intended to limit the spirit or scope thereof. It will be understood that the embodiments of the present disclosure described herein are merely exemplary and that a person skilled in the art can make any variations and modification without departing from the spirit and scope of the disclosure. All such variations and modifications, including those discussed above, are intended to be included within the scope of the disclosure.

1. A health pass system for COVID-19, comprising:
a database including information on a plurality of individuals;
the database configured to receive point-of-care data relating to the plurality of individuals, the point-of-care data including at least one medical test result for COVID-19 antibodies for at least one of the plurality of individuals;
a processing device configured to analyze the point-of-care data and generate a report;
means for transmitting the report to a mobile electronic device of at least one of the plurality of individuals; and
means for displaying the report with a user interface of the mobile electronic device in the form of a real-time health certification, the report including (i) the at least one medical test result for COVID-19 antibodies for the at least one individual, and (ii) an indication of a positive or negative result of the at least one medical test result for COVID-19 antibodies.

2. The health report system of claim 1, wherein the report includes a code capable of being scanned to display information associated with the report on another user interface or an electronic device, and wherein the code is a QR code.

3. The health report system of claim 1, wherein the report includes a photograph of the individual.

4. The health report system of claim 1, wherein the report includes a unique alphanumeric identification associated with the individual.

5. The health report system of claim 1, wherein the report includes a name of the individual, a type of test performed for the at least one medical test result, and a date of the test performed for the at least one medical test result.

6. The health report system of claim 1, wherein the point-of-care data includes at least one of manual laboratory test results, automatic laboratory test results, medical information, private test results, or mandated test results.

7. The health report system of claim 1, wherein the point-of-care data includes information relating to or derived from at least one of DNA, antibodies, psychological evaluation, existing conditions, medical history, plasma, blood composition, saliva, bodily fluid, stool, bone marrow, enzymes, glucose, cholesterol, immunology, gastric fluid, kidney function, inulin, pregnancy, toxicology, urinalysis, thyroid function, genetic testing, or skin testing.

8. The health report system of claim 1, wherein the at least one medical test result for COVID-19 antibodies includes IGG+ and IGM− antibodies.

9. The health report system of claim 1, wherein the database is configured to receive the point-of-care data from medical tests in real-time.

10. The health report system of claim 1, wherein the database is configured to receive the point-of-care data from already administered medical tests.

11. The health report system of claim 1, wherein the database is configured to receive the point-of-care data from an automated testing system or a photograph of a medical test result received as input.

12. The health report system of claim 1, wherein the processing device is configured to consolidate the point-of-care data and convert the point-of-care data into a common storage format having a unified data structure.

13. The health report system of claim 1, wherein the processing device is configured to generate a risk score based on analysis of the point-of-care data, the risk score indicating one or more biological risks associated with the plurality of individuals.

14. The health report system of claim 1, wherein the processing device is configured to generate a risk score based on the point-of-care data and medical guidelines including thresholds for the point-of-care data regarding biological illness levels or risks.

15. The health report system of claim 13, wherein the processing device is configured to automatically update the risk score based on real-time updated medical guidelines.

16. The health report system of claim 13, wherein the processing device is configured to generate the risk score based on the point-of-care data, prior conditions, demographics, and data collected at a point of presence.

17. The health report system of claim 13, wherein the report includes the risk score.

18. The health report system of claim 13, wherein based on the generated risk score, the report includes a designation of safe or note safe for the at least one of the plurality of individuals.

19. A method of generating a health pass for COVID-19, the method comprising:
receiving at a database information a plurality of individuals;
receiving at the database point-of-care data relating to the plurality of individuals, the point-of-care data including at least one medical test result for COVID-19 antibodies for at least one of the plurality of individuals;
analyzing the point-of-care data and generating a report with a processing device;
transmitting the report to a mobile electronic device of at least one of the plurality of individuals; and
displaying the report with a user interface of the mobile electronic device in the form of a real-time health certification, the report including (i) the at least one medical test result for COVID-19 antibodies for the at least one individual, (ii) an indication of a positive or negative result of the at least one medical test result for COVID-19 antibodies, and (iii) a code capable of being scanned to display information associated with the report on another user interface or an electronic device.

20. A non-transitory computer-readable medium storing instructions at least for generating a health pass for COVID-19 that are executable by a processing device, wherein execution of the instructions by the processing device causes the processing device to:
receive at a database information a plurality of individuals;
receive at the database point-of-care data relating to the plurality of individuals, the point-of-care data including at least one medical test result for COVID-19 antibodies for at least one of the plurality of individuals;
analyze the point-of-care data and generate a report;
transmit the report to a mobile electronic device of at least one of the plurality of individuals; and
display the report with a user interface of the mobile electronic device in the form of a real-time health certification, the report including (i) the at least one medical test result for COVID-19 antibodies for the at least one individual, (ii) an indication of a positive or negative result of the at least one medical test result for COVID-19 antibodies, and (iii) a code capable of being scanned to display information associated with the report on another user interface or an electronic device.
